(19) United States
(12) Patent Application Publication
(10) Pub. No.: US 2026/0114769 A1
(43) Pub. Date: Apr. 30, 2026

(54) METHOD AND APPARATUS FOR DETERMINING DEPRESSION AND ANALYZING MENTAL HEALTH BASED ON MULTIMODAL ARTIFICIAL INTELLIGENCE

(71) Applicant: ELECTRONICS AND TELECOMMUNICATIONS RESEARCH INSTITUTE, Daejeon (KR)

(72) Inventors: Aram Lee, Daejeon (KR); Sehwan MOON, Daejeon (KR); Eun Kyoung Jeon, Daejeon (KR); Jeong Eun Kim, Daejeon (KR)

(21) Appl. No.: 19/375,602

(22) Filed: Oct. 31, 2025

(30) Foreign Application Priority Data
Oct. 31, 2024 (KR) 10-2024-0152221
Apr. 15, 2025 (KR) 10-2025-0048530

(51) Int. Cl. A61B 5/16; A61B 5/00; A61B 5/0205; A61B 5/024; A61B 5/318; G10L 25/66; G16H 50/20; G16H 50/30

(52) U.S. Cl. CPC A61B 5/165; A61B 5/0205; A61B 5/318; A61B 5/4803; A61B 5/4842; G10L 25/66; G16H 50/20; G16H 50/30; A61B 5/02405

(57) ABSTRACT

Disclosed are a method and apparatus for determining depression and analyzing mental health based on multimodal AI. The apparatus includes a multimodal data collection unit that collects multimodal data including voice data, text data recognized from the verbal output, and ECG data of a user, a depression state determination unit that outputs depression state data indicating a depression state of the user by using an AI model based on the multimodal data, a medical data and biometric information association unit that collects a physical health index by associating biometric information including medical data, blood pressure, body composition measurements, and arrhythmia measurement results of the user, a mental health analysis unit that generates mental health analysis results from the multimodal data, the depression state data, and the physical health index by using LLM, and an analysis result generation unit that visually provides the user with the mental health analysis results.

### CROSS-REFERENCE TO RELATED APPLICATION(S)

This application claims priority from and the benefit of Korean Patent Application No. 10-2024-0152221, filed on Oct. 31, 2024, and Korean Patent Application No. 10-2025-0048530, filed on Apr. 15, 2025, which are hereby incorporated by reference for all purposes as if set forth herein.

### BACKGROUND

### 1. Technical Field

The present disclosure relates to a method and apparatus for determining depression and analyzing mental health based on multimodal artificial intelligence.

### 2. Description of Related Art

A conventional mental health monitoring technique has limitations in performing the comprehensive analysis of mental health because the technique basically checks an emotional state based on single-modality data, such as text or voices. Furthermore, a conventional method has difficulty in accurately checking an emotional change or state in real time because the method depends on the self-reporting of a user.

Recently, attempts to comprehensively evaluate emotional and physical health conditions by analyzing various biometric data, such as heart rate variability (HRV) and activity are increased. There is a need for a system for accurately analyzing a personal health condition by efficiently integrating such multimodal data and automatically generating the results of the analysis.

### SUMMARY

Various embodiments are directed to providing a method and apparatus for determining depression and analyzing mental health based on multimodal artificial intelligence (AI), which can provide the analysis of mental health having high reliability based on multimodal data.

An apparatus for determining depression and analyzing mental health according to an embodiment of the present disclosure includes a multimodal data collection unit configured to collect multimodal data including voice data of a user, text data recognized from an verbal output of the user, and electrocardiogram (ECG) data of the user, a depression state determination unit configured to output depression state data indicative of a depression state of the user by using an artificial intelligence (AI) model based on the collected multimodal data, a medical data and biometric information association unit configured to collect a physical health index by associating biometric information including medical data, blood pressure, body composition measurements, and arrhythmia measurement results of the user, a mental health analysis unit configured to generate mental health analysis results from the multimodal data, the depression state data, and the physical health index by using a large language model (LLM), and an analysis result generation unit configured to visually provide the user with the mental health analysis results generated by the mental health analysis unit.

In an embodiment, the multimodal data collection unit may include a voice feature extraction unit configured to extract voice feature data from the voice data and an ECG feature extraction unit configured to extract heart rate feature data from the ECG data.

In an embodiment, the heart rate feature data may include heart rate variability.

In an embodiment, the depression state determination unit includes a first neural network configured to process the voice feature data, a second neural network configured to process the text data, a third neural network configured to process the heart rate feature data, and a multi-modal processing unit configured to calculate the depression state data based on output values from the first to third neural networks. In an embodiment, the depression state determination unit outputs a number indicative of the severity of depression as the depression state data.

In an embodiment, the mental health analysis unit generates a prompt through prompt optimization and generates personalized health analysis results by inputting the generated prompt into the LLM.

In an embodiment, the analysis result generation unit comprehensively analyzes two or more data through prompt optimization based on few-shot chain-of-thought (CoT) and generates a prompt in an HTML form by identifying core information of the results of the analysis of the two or more data.

In an embodiment, the text collected by the multimodal data collection unit may include postings on a social network service or a piece of writing completed on a questionnaire by the user.

In an embodiment, the data collected by the multimodal data collection unit includes activity data. The medical data and biometric information association unit outputs a visualized image after circadian-fitting the activity data.

An apparatus for analyzing mental health according to an embodiment of the present disclosure includes a multimodal data collection unit configured to collect multimodal data including voice data of a user, text data recognized from an verbal output of the user, and electrocardiogram (ECG) data of the user, a medical data and biometric information association unit configured to collect a physical health index by associating biometric information including medical data, blood pressure, body composition measurements, and arrhythmia measurement results of the user, a mental health analysis unit configured to generate mental health analysis results from the multimodal data and the physical health index by using a large language model (LLM), and an analysis result generation unit configured to visually provide the user with the mental health analysis results generated by the mental health analysis unit.

In an embodiment, the multimodal data collection unit includes a voice feature extraction unit configured to extract voice feature data from the voice data and an ECG feature extraction unit configured to extract heart rate feature data from the ECG data.

In an embodiment, the mental health analysis unit generates a prompt through prompt optimization and generates personalized health analysis results by inputting the generated prompt into the LLM.

In an embodiment, the analysis result generation unit comprehensively analyzes two or more data through prompt optimization based on few-shot chain-of-thought (CoT) and generates a prompt in an HTML form by identifying core information of the results of the analysis of the two or more data.

In an embodiment, the text collected by the multimodal data collection unit may include postings on a social network service or a piece of writing completed on a questionnaire by the user.

In an embodiment, the data collected by the multimodal data collection unit may include activity data. The medical data and biometric information association unit outputs a visualized image after circadian-fitting the activity data.

A method of determining depression and analyzing mental health according to an embodiment of the present disclosure includes a multimodal data collection step of collecting multimodal data including voice data of a user, text data recognized from an verbal output of the user, and electrocardiogram (ECG) data of the user, a depression state determination step of outputting depression state data indicative of a depression state of the user by using an artificial intelligence (AI) model based on the collected multimodal data, a medical data and biometric information association step of collecting a physical health index by associating biometric information including medical data, blood pressure, body composition measurements, and arrhythmia measurement results of the user, a mental health analysis step of generating mental health analysis results from the multimodal data, the depression state data, and the physical health index by using a large language model (LLM), and an analysis result generation step of visually providing the user with the mental health analysis results generated in the mental health analysis step.

In an embodiment, the multimodal data collection step includes a voice feature extraction step of extracting voice feature data from the voice data and a heart rate feature extraction step of extracting heart rate feature data from the ECG data.

In an embodiment, the depression state determination step includes a step of processing the voice feature data by using a first neural network, a step of processing the text data by using a second neural network, a step of processing the heart rate feature data by using a third neural network, and a multi-modal processing step of calculating the depression state data based on output values from the first to third neural networks.

In an embodiment, the mental health analysis step includes steps of generating a prompt through prompt optimization and generating personalized health analysis results by inputting the generated prompt into the LLM.

In an embodiment, the analysis result generation step includes steps of comprehensively analyzing two or more data through prompt optimization based on few-shot chain-of-thought (CoT) and generating a prompt in an HTML form by identifying core information of the results of the analysis of the two or more data.

According to embodiments of the present disclosure, a highly reliable mental health analysis can be provided because the depression state of a user is determined based on emotional information (e.g., memories of happiness and unhappiness) extracted from a spontaneous verbal output of the user, such as voices or text, and the ECG data of the user and the results of the analysis of personalized mental health are additionally provided by integrating various biometric data, such as activity, medical MyData, sleep, blood pressure, body composition, and arrhythmia measurement results. Furthermore, a user can be supported to understand his or her own emotional and physical health conditions more accurately and take necessary measures because the user is provided with comprehensive metal health analysis results that are automatically generated by a large language model (LLM) using a prompt engineering technique.

Effects of the present disclosure are not limited to the aforementioned effects, and the other effects not described above may be evidently understood by a person having ordinary knowledge in the art from the following description.

### BRIEF DESCRIPTION OF THE DRAWINGS

FIG. 1 is a block diagram illustrating a construction of an apparatus for determining depression and analyzing mental health based on multimodal artificial intelligence (AI) according to an embodiment of the present disclosure.

FIG. 2 illustrates some examples of feature data that are extracted from voice data.

FIG. 3 is a graph illustrating intervals between continuous heart beats that are used to calculate heart rate variability.

FIG. 4 illustrates an example in which a depression state determination unit calculates depression state data based on data collected by a multimodal data collection unit.

FIG. 5 illustrates an example of data that are input to a mental health analysis unit.

FIG. 6 illustrates an example in which a medical data and biometric information association unit associates medical data and biometric information.

FIG. 7 illustrates an example of a prompt that is generated by the mental health analysis unit.

FIG. 8 illustrates an example of the results of analysis that are output by an analysis result generation unit.

### DETAILED DESCRIPTION

The aforementioned object, other objects, advantages, and characteristics of the present disclosure and a method for achieving the objects, advantages, and characteristics will become clear with reference to embodiments to be described in detail along with the accompanying drawings.

However, the present disclosure is not limited to embodiments disclosed hereinafter, but may be implemented in various different forms. The following embodiments are merely provided to easily notify a person having ordinary knowledge in the art to which the present disclosure pertains of the objects, constructions, and effects of the present disclosure. The scope of rights of the present disclosure is defined by the writing of the claims.

Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other components, steps, operations and/or components in addition to mentioned components, steps, operations and/or components.

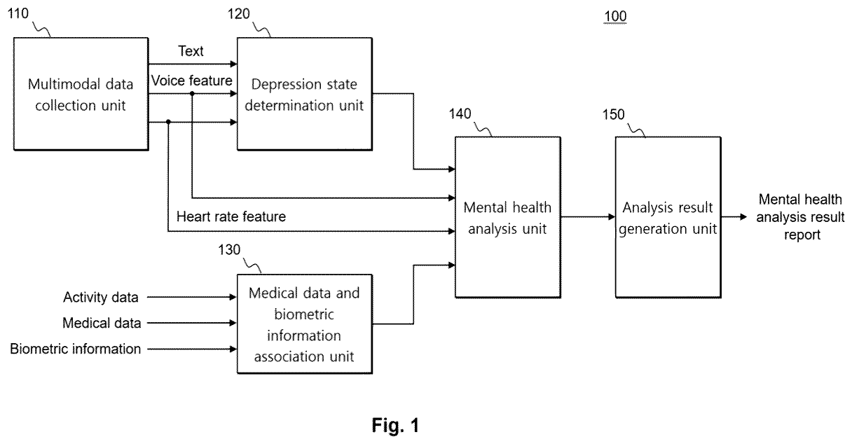

FIG. 1 is a block diagram illustrating a construction of an apparatus for determining depression and analyzing mental health based on multimodal artificial intelligence (AI) according to an embodiment of the present disclosure. The apparatus for determining depression and analyzing mental health based on multimodal AI in FIG. 1 includes a multimodal data collection unit 110, a depression state determination unit 120, a medical data and biometric information association unit 130, a mental health analysis unit 140 based on prompt engineering and large language model (LLM), and an analysis result generation unit 150.

The multimodal data collection unit 110 collects the voice data of a user, text recognized from the verbal output of the user, electrocardiogram (ECG), and activity data. In an embodiment, the voice and text data are used to analyze emotional information including memories of happiness and unhappiness. In an embodiment, postings on a social network service (SNS) or a piece of writing completed on a questionnaire by a user may be used as the text data.

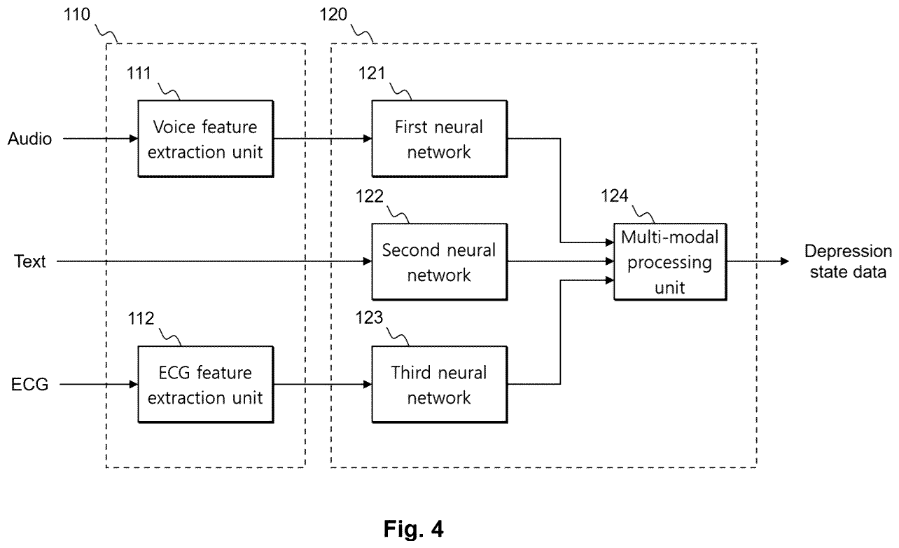

As illustrated in FIG. 4, the multimodal data collection unit 110 includes a voice feature extraction unit 111 that extracts voice feature data from voice data and an ECG feature extraction unit 112 that extracts heart rate feature data from ECG data.

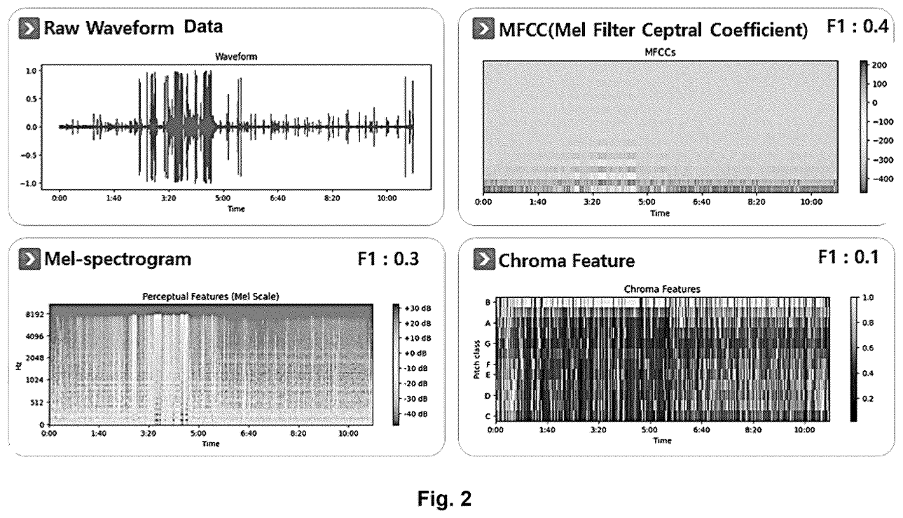

FIG. 2 illustrates some examples of feature data that are extracted from voice data. In the case of voices, as in the example of FIG. 2, several tens to several thousands of features are extracted from recorded digital voice data. Representative features include the tone, tremor, and intensity for each frequency band, of a voice. The tendency of features according to the emotional state is learnt through machine learning. In the case of more detailed frequency analysis, after a frequency spectrum is imaged, the frequency spectrum may be learnt through a convolutional neural network (CNN).

Text data are most basically used to monitor whether positive expressions or negative expressions are used. According to embodiments, although enantiosis is used, a speaker's intention may be checked by analyzing the entire context.

ECG data are used to analyze the emotional state more finely by extracting heart rate variability (HRV) features.

The heart rate indicates how many times the heart beats per minute. Heart rate variability indicates how irregular the heart rate's periodicity is. Human's ECG is controlled by an autonomic nervous system. Feedback control is applied to the ECG according to the emotional state. Accordingly, the autonomic nervous system indicates that the ECG is actively changed in the case of a healthy person, and indicates that the ECG is very consistent in the case of an unhealthy person. That is, in the case of physically unstable people, the ECG has a constant pattern and low heart rate variability because the autonomic nervous system is weakened. Accordingly, when the heart rate variability is low, a corresponding person may be determined to be physically unstable.

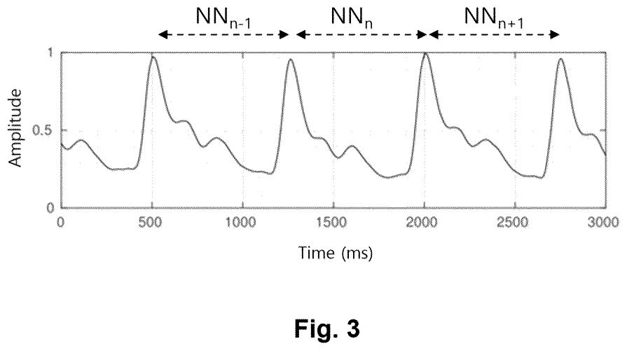

The heart rate variability may be expressed as several tens of parameters in time, frequency, and non-linear domains. In an embodiment, in order to calculate the heart rate variability, as illustrated in FIG. 3, NN-intervals NNₙ₋₁, NNₙ, NNₙ₊₁, . . . between continuous ECGs are calculated and statistically analyzed.

For example, standard deviation of NN intervals (SDNN), that is, a standard deviation of NN-intervals, may be used. In addition to the SDNN, standard deviation of RR intervals (SDRR), standard deviation of the average NN intervals for each 5 min segment of a 24h HRV recording (SDANN), percentage of successive RR intervals that differ by more than 50 ms (pNN50), average difference between the highest and lowest heart rates during each respiratory cycle (HR Max-HR Min), root mean square of successive RR interval differences (RMSSD), integral of the density of RR interval histogram divided by its height (HRV triangular index), and baseline width of the RR interval histogram (TINN) may be used to calculate the heart rate variability. The NN interval is an interval between ECGs after artifacts are removed. The RR interval is an interval between all of successive ECGs.

The voice feature data, text data, and heart rate feature data collected by the multimodal data collection unit 110 are transmitted to the depression state determination unit 120. The depression state determination unit 120 determines the depression state of the user by using an artificial intelligence (AI) model based on the collected multimodal data.

As illustrated in FIG. 4, the depression state determination unit 120 includes a first neural network 121 that processes the voice feature data extracted from the voice feature extraction unit 111, a second neural network 122 that processes the text data collected by the multimodal data collection unit 110, a third neural network 123 that processes the heart rate feature data extracted from the ECG feature extraction unit 112, and a multi-modal processing unit 124 that calculates depression state data based on output values from the first to third neural networks 121, 122, and 123. The multi-modal processing unit 124 may also be implemented as an AI model.

Each of the first to third AI models 121, 122, and 123 learns relevance with mental health by analyzing features extracted from user clinical data having various modalities. In an embodiment, the AI model may output the depression state of a user as 0 (normal) and 1 (depression) by classifying the depression state in a binary form. In another embodiment, the AI model may classify the severity of depression in a quad form.

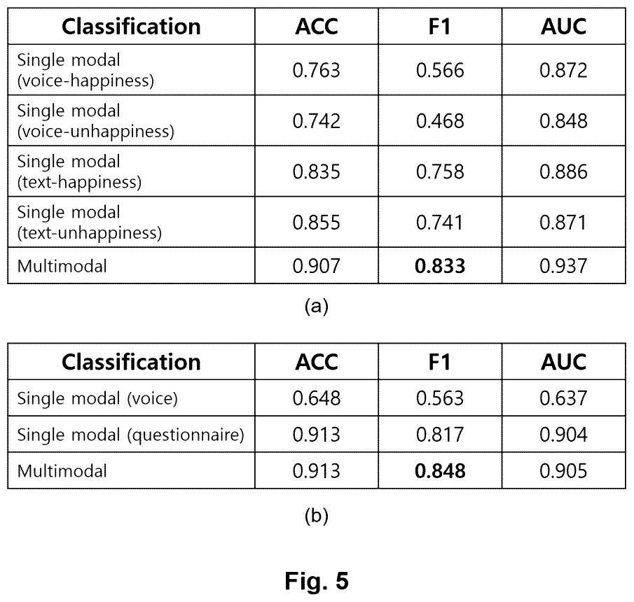

Examples of the output depression state data values are illustrated in FIG. 5. In the example of FIG. 4, each of the first to third AI models 121, 122, and 123 calculates primary data from each of input modality data. The multi-modal processing unit 124 outputs a final multimodal depression state data value by inputting the primary data to each AI model. In FIG. 5(a), after values indicative of the level of happiness and the level of unhappiness are derived from single-modal data, the multi-modal processing unit 124 outputs a final depression state data value. In FIG. 5(b), after a feature value indicative of the size of depression is derived from single-modal data, the multi-modal processing unit 124 outputs a final depression state data value based on the feature value.

As described above, the depression state determination unit 120 may output a numerical value representing the severity of depression as a depression state data value. The depression state data output by the depression state determination unit 120 are used as one of inputs to the mental health analysis unit 140. When the depression state data value is high, the mental health analysis unit 140 may write mental health analysis text including contents indicating that the depression state of a user is severe. For example, when the range of the depression state data value is 0 to 1, if the level of depression is determined to be 0.8, the mental health analysis unit 140 may output results, such as “The current level of depression has been shown to be severe. This indicates that the health of the mind is very tired, and the current state may have a big impact on daily life and emotional control.”

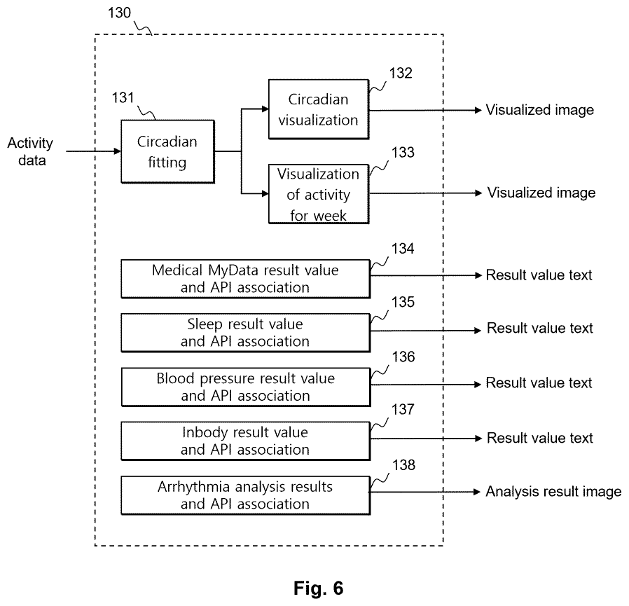

The medical data and biometric information association unit 130 collects a physical health index by associating the medical data, sleep, blood pressure, body composition measurements, and arrhythmia measurement results of a user in addition to the activity data of the user so that an overall health condition can be comprehensively analyzed based on the physical health index. FIG. 6 illustrates an example in which the medical data and biometric information association unit associates medical data and biometric information.

In an embodiment, the medical data and biometric information association unit 130 outputs a visualized image from the activity data of a user. To this end, after circadian-fitting the activity data (131), the medical data and biometric information association unit 130 may generate the visualized image by circadian-visualizing the activity data (132). Furthermore, the medical data and biometric information association unit 130 may be configured to visualize activity for a week (133) from the circadian-fitted data.

Furthermore, the medical data and biometric information association unit 130 may associate data as follows.
Medical MyData result value and API association (134): PHR association. A person directly downloads a file according to Act on the Protection of Personal Information and uploads a PDF file.
Sleep result value and API association (135): a sleep sensor and an API are associated. Data are associated with a platform through the association of member information.
Blood pressure result value and API association (136): data measured by a blood pressure measuring device are associated with the platform through the association of member information as a QR code after personal information consent.
Body composition analyzer (inbody) result value and API association (137): a result value measured by a body composition analyzer is associated with the platform through the association of a body composition member number after personal information consent.
Arrhythmia analysis results and API association (138): a person directly uploads an image of the results of the analysis of another company for which a medical device has been authorized.

A method of associating data may be different according to the policy of a measuring device manufacturer, such as a blood pressure gauge, a sleep sensor, a body composition analyzer, or an arrhythmia measuring device.

The mental health analysis unit 140 generates personalized health analysis results from the multimodal data collected by the multimodal data collection unit 110, the depression state data output by the depression state determination unit 120, and the physical health index output by the medical data and biometric information association unit 130 by using a large language model (LLM). The mental health analysis unit 140 generates a prompt from the collected multimodal data and the physical health index, and transmits the generated prompt as an input value for the LLM. The LLM analyzes body and mental health conditions from the input prompt and the multimodal data and generates the personalized health analysis results by synthesizing the body and mental health conditions.

In an embodiment, the mental health analysis unit 140 may generate results by analyzing each of data based on prompt optimization based on few-shot chain of tree (CoT). Data which may be analyzed include voices, activity, heart rate variability, text, medical MyData, sleep data, blood pressure data, body composition data, and arrhythmia data.

The prompt using the prompt optimization based on few-shot CoT, which is generated by the mental health analysis unit 140 may include a task that describes detailed task contents to be performed, a format that describes the structure or layout of information and an answer to which reference needs to be made upon analysis, and an example that describes an example of analysis results, that is, a detailed example of desired results.

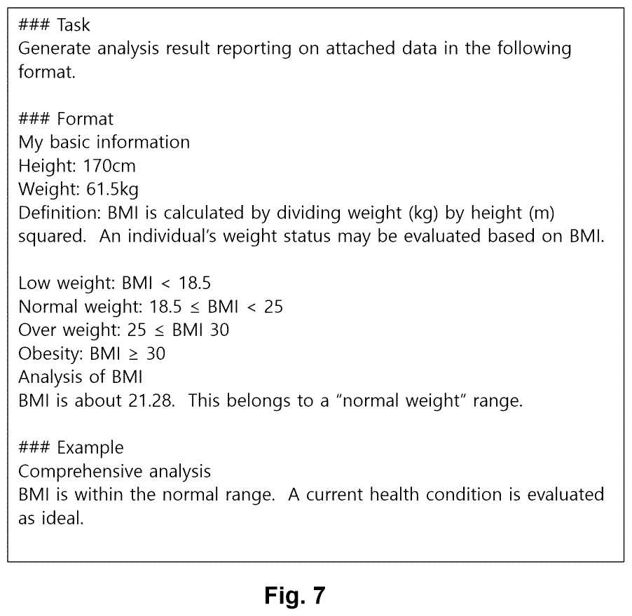

FIG. 7 illustrates an example of a prompt that is generated by the mental health analysis unit 140. FIG. 7 is an example of the prompt for analyzing a body mass index. The mental health analysis unit 140 outputs analysis results related to body mass by inputting the prompt to an LLM. A prompt, such as the example of FIG. 7, may be generated with respect to each of voices, activity, heart rate variability, text, medical MyData, sleep data, blood pressure data, body composition data, and arrhythmia data. The results of the analysis of each of the voices, activity, heart rate variability, text, medical MyData, sleep data, blood pressure data, body composition data, and arrhythmia data may be output by inputting the prompt into the LLM.

The analysis result generation unit 150 visually provides a user with mental health analysis results that are finally generated by the mental health analysis unit 140. The analysis result generation unit 150 provides the user with the mental health analysis results that are automatically generated by the mental health analysis unit 140 so that the user can easily understand the mental health analysis results. In an embodiment, the analysis result generation unit 150 may visualize the mental health analysis results by using a predetermined template and provide a personalized advice.

In an embodiment, the analysis result generation unit 150 comprehensively analyzes two or more data through prompt optimization based on few-shot chain-of-thought (CoT) and generates a prompt in an HTML form by identifying core information of the results of the analysis of the two or more data. That is, the analysis result generation unit 150 generates a prompt through prompt optimization based on few-shot CoT and transmits the generated prompt as an input value for an LLM. The LLM generates a mental health analysis result report having an HTML form.

The prompt through prompt optimization based on few-shot CoT, which is generated by the analysis result generation unit 150, may include Fersona that includes a description of what module is being analyzed, a task that describes the details of work to be performed by writing and guiding an analysis method for each procedure as if introducing a flow of thoughts, a format that describes the structure or layout of information and an answer to be referenced upon analysis, and an example that describes an example of the results of analysis, that is, a detailed example of desired results. The example may include an HTML format of the mental health analysis result report, for example. According to embodiments, Fersona may be omitted. An example of a task that generates the mental health analysis result report is as follows.
1. Please write a general review within about 20 lines on the basis of six analysis results included in input data.
2. Please provide a general review on the basis of contents included in the input data when the six analysis results are not present.
3. Please subdivide the general review by region and present the subdivided reviews in a form in which only problematic parts are emphasized again.
4. For example, areas with a high risk level may be highlighted, and it may be suggested that the user seek professional consultation along with the corresponding numerical values.
5. Please mark the most important part in bold and change colors in the general review.
6. Please write overall evaluation in an HTML structure.
7. Please generate a general review for each user.

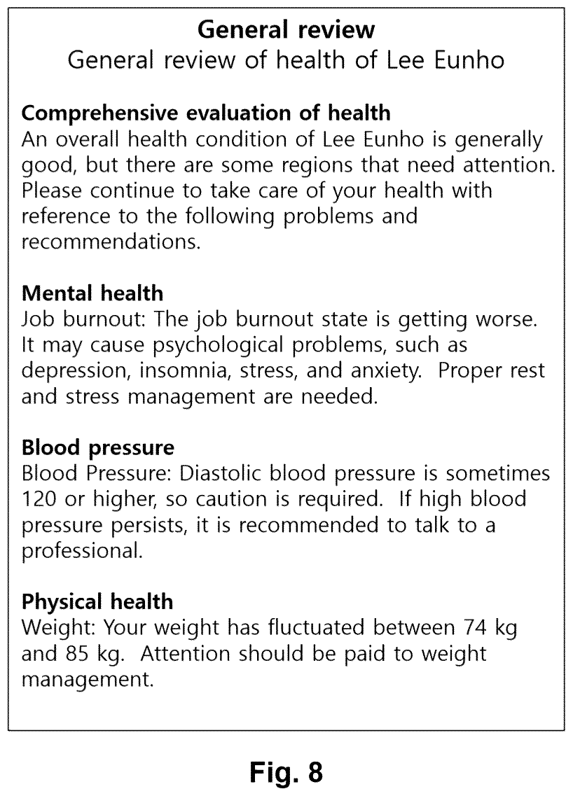

FIG. 8 illustrates an example of the results of analysis that are output by the analysis result generation unit 150. As illustrated in FIG. 8, the analysis result generation unit 150 generates core information of analysis results in the HTML form by identifying the core information and displays the core information at a front end.

The method according to an embodiment of the present disclosure may be implemented in the form of a program instruction which may be executed through various computer means, and may be recorded on a computer-readable medium.

The computer-readable medium may include a program instruction, a data file, and a data structure alone or in combination. A program instruction recorded on the computer-readable medium may be specially designed and constructed for an embodiment of the present disclosure or may be known and available to those skilled in the computer software field. The computer-readable medium may include a hardware device configured to store and execute the program instruction. For example, the computer-readable medium may include magnetic media such as a hard disk, a floppy disk, and a magnetic tape, optical media such as CD-ROM and a DVD, magneto-optical media such as a floptical disk, ROM, RAM, and flash memory. The program instruction may include not only a machine code produced by a compiler, but a high-level language code capable of being executed by a computer through an interpreter.

The embodiments of the present disclosure have been described in detail, but the scope of rights of the present disclosure is not limited thereto. A variety of modifications and changes made by those skilled in the art using the basic concept of the present disclosure defined in the appended claims are also included in the scope of rights of the present disclosure.

### DESCRIPTION OF REFERENCE NUMERALS

110: multimodal data collection unit, 120: depression state determination unit, 130: medical data and biometric information association unit, 140: mental health analysis unit, 150: analysis result generation unit

What is claimed is:

1. An apparatus for determining depression and analyzing mental health, the apparatus comprising:
a multimodal data collection unit configured to collect multimodal data comprising voice data of a user, text data recognized from an verbal output of the user, and electrocardiogram (ECG) data of the user;
a depression state determination unit configured to output depression state data indicative of a depression state of the user by using an artificial intelligence (AI) model based on the collected multimodal data;
a medical data and biometric information association unit configured to collect a physical health index by associating biometric information comprising medical data, blood pressure, body composition measurements, and arrhythmia measurement results of the user;
a mental health analysis unit configured to generate mental health analysis results from the multimodal data, the depression state data, and the physical health index by using a large language model (LLM); and
an analysis result generation unit configured to visually provide the user with the mental health analysis results generated by the mental health analysis unit.

2. The apparatus of claim 1, wherein the multimodal data collection unit comprises:
a voice feature extraction unit configured to extract voice feature data from the voice data, and
an ECG feature extraction unit configured to extract heart rate feature data from the ECG data.

3. The apparatus of claim 2, wherein the heart rate feature data comprise heart rate variability.

4. The apparatus of claim 2, wherein the depression state determination unit comprises:
a first neural network configured to process the voice feature data;
a second neural network configured to process the text data;
a third neural network configured to process the heart rate feature data; and
a multi-modal processing unit configured to calculate the depression state data based on output values from the first to third neural networks.

5. The apparatus of claim 2, wherein the depression state determination unit outputs a number indicative of a severity of depression as the depression state data.

6. The apparatus of claim 1, wherein the mental health analysis unit generates a prompt through prompt optimization and generates personalized health analysis results by inputting the generated prompt into the LLM.

7. The apparatus of claim 1, wherein the analysis result generation unit comprehensively analyzes two or more data through prompt optimization based on few-shot chain-of-thought (CoT) and generates a prompt in an HTML form by identifying core information of the results of the analysis of the two or more data.

8. The apparatus of claim 1, wherein the text collected by the multimodal data collection unit comprises postings on a social network service or a piece of writing completed on a questionnaire by the user.

9. The apparatus of claim 1, wherein:
the data collected by the multimodal data collection unit comprise activity data, and
the medical data and biometric information association unit outputs a visualized image after circadian-fitting the activity data.

10. An apparatus for analyzing mental health, comprising:
a multimodal data collection unit configured to collect multimodal data comprising voice data of a user, text data recognized from an verbal output of the user, and electrocardiogram (ECG) data of the user;
a medical data and biometric information association unit configured to collect a physical health index by associating biometric information comprising medical data, blood pressure, body composition measurements, and arrhythmia measurement results of the user;
a mental health analysis unit configured to generate mental health analysis results from the multimodal data and the physical health index by using a large language model (LLM); and
an analysis result generation unit configured to visually provide the user with the mental health analysis results generated by the mental health analysis unit.

11. The apparatus of claim 10, wherein the multimodal data collection unit comprises:
a voice feature extraction unit configured to extract voice feature data from the voice data, and
an ECG feature extraction unit configured to extract heart rate feature data from the ECG data.

12. The apparatus of claim 10, wherein the mental health analysis unit generates a prompt through prompt optimization and generates personalized health analysis results by inputting the generated prompt into the LLM.

13. The apparatus of claim 10, wherein the analysis result generation unit comprehensively analyzes two or more data through prompt optimization based on few-shot chain-of-thought (CoT) and generates a prompt in an HTML form by identifying core information of the results of the analysis of the two or more data.

14. The apparatus of claim 10, wherein the text collected by the multimodal data collection unit comprises postings on a social network service or a piece of writing completed on a questionnaire by the user.

15. The apparatus of claim 10, wherein:
the data collected by the multimodal data collection unit comprise activity data, and
the medical data and biometric information association unit outputs a visualized image after circadian-fitting the activity data.

16. A method of determining depression and analyzing mental health, the method comprising:
a multimodal data collection step of collecting multimodal data comprising voice data of a user, text data recognized from an verbal output of the user, and electrocardiogram (ECG) data of the user;
a depression state determination step of outputting depression state data indicative of a depression state of the user by using an artificial intelligence (AI) model based on the collected multimodal data;
a medical data and biometric information association step of collecting a physical health index by associating biometric information comprising medical data, blood pressure, body composition measurements, and arrhythmia measurement results of the user;
a mental health analysis step of generating mental health analysis results from the multimodal data, the depression state data, and the physical health index by using a large language model (LLM); and
an analysis result generation step of visually providing the user with the mental health analysis results generated in the mental health analysis step.

17. The method of claim 16, wherein the multimodal data collection step comprises:
a voice feature extraction step of extracting voice feature data from the voice data, and
a heart rate feature extraction step of extracting heart rate feature data from the ECG data.

18. The method of claim 17, wherein the depression state determination step comprises:
a step of processing the voice feature data by using a first neural network;
a step of processing the text data by using a second neural network;
a step of processing the heart rate feature data by using a third neural network; and
a multi-modal processing step of calculating the depression state data based on output values from the first to third neural networks.

19. The method of claim 16, wherein the mental health analysis step comprises steps of:
generating a prompt through prompt optimization, and
generating personalized health analysis results by inputting the generated prompt into the LLM.

20. The method of claim 16, wherein the analysis result generation step comprises steps of:
comprehensively analyzing two or more data through prompt optimization based on few-shot chain-of-thought (CoT), and
generating a prompt in an HTML form by identifying core information of the results of the analysis of the two or more data.
